(19) United States
(12) Patent Application Publication
(10) Pub. No.: US 2019/0099383 A1
(43) Pub. Date: Apr. 4, 2019

(54) Transdermal Delivery of Selexipag Metabolite

(71) Applicant: Agis Kydonieus, Kendall Park, NJ (US)

(72) Inventor: Agis Kydonieus, Kendall Park, NJ (US)

(73) Assignee: Samos Pharmaceuticals, Inc., Kendall Park, NJ (US)

(21) Appl. No.: 16/146,407

(22) Filed: Sep. 28, 2018

Related U.S. Application Data
(60) Provisional application No. 62/606,635, filed on Sep. 30, 2017.

(51) Int. Cl. A61K 9/70; A61K 31/4965; A61K 47/32; A61K 47/34; A61K 9/00; A61K 9/06; A61K 9/107; A61K 47/38

(52) U.S. Cl. CPC A61K 9/7084; A61K 31/4965; A61K 47/32; A61K 47/34; A61K 47/38; A61K 9/06; A61K 9/107; A61K 9/1075; A61K 9/0014

(57) ABSTRACT

Compositions, devices and methods for the transdermal delivery of drugs that are potent vasorelaxants and inhibitors of human platelet aggregation are disclosed. These drugs bind to the IP receptor on endothelial and platelet cells and they are useful in the treatment of pulmonary arterial hypertension and other diseases where vasoconstriction is an issue. The compositions, methods and devices pertain particularly to the transdermal delivery of the selexipag metabolite ACT 333679.

### CROSS-REFERENCE TO RELATED APPLICATIONS

This claims benefit of U.S. Provisional Application No. 62/606,635, filed Sep. 30, 2017, the entire contents of which are incorporated by reference herein.

### FIELD OF THE INVENTION

The invention pertains to the transdermal delivery of drugs that are potent vasorelaxants and inhibitors of human platelet aggregation. The invention more particularly pertains to compositions and methods for the transdermal delivery of the selexipag metabolite ACT 333679.

### BACKGROUND

Selexipag was approved in the United States in 2015 for the treatment of pulmonary arterial hypertension (PAH) and to prolong disease progression. PAH is characterized by pulmonary vasoconstriction, vascular cell proliferation and vascular hypertrophy leading to an increase in pulmonary artery pressure, right ventricular hypertrophy and right heart failure. Selexipag and its active metabolite ACT 333679 also known as MRE 269 act as selective agonists of the prostacyclin receptor to increase vasodilation in the pulmonary circulation and decrease the elevated pressure in the blood vessels supplying blood to the lungs. The drug product is administered orally in immediate release tablets containing from 200 micrograms selexipag and up to 1600 micrograms, in increasing dose values of 200 microgram. The drug administration is bid (twice per day) because of the low half-life of selexipag as well as of the metabolite ACT 333679. Selexipag is partially metabolized in the liver by carboxylate 1 to form the active metabolite ACT 333679 which is at least 16 times more potent than selexipag and it is present at 3 to 4 fold higher plasma concentrations than selexipag in the human plasma at steady state. Below I present some of the background information that could be pertinent to transdermal delivery, and a complete review of the selexipag assessment can be found in the report, European Medicines Agency/Uptravi Assessment Report—EMA/272184/2016.

The chemical structures of selexipag and ACT 333679 and the conversion by hydrolysis of selexipag to ACT 333679 is shown below:

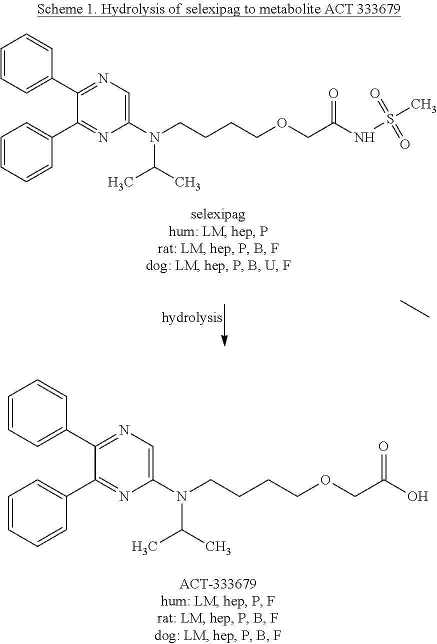

Pharmacokinetics: The pharmacokinetics of selexipag were mainly performed in animals and showed rapid absorption with oral bioavailability of ACT 333679 between 29 to 57%. ACT 333679 was also rapidly absorbed in the rat with an oral bioavailability of 57%. ACT 333679 has much higher activity than selexipag for the prostacyclin IP receptor and thus it is at least 16 times more potent than selexipag and it is present in the plasma at 3 to 4 fold higher levels at steady state. The half-life of selexipag is very low at 0.8 to 2.5 hours and that of the metabolite 6 to 13 hours. Presence of food also affected the AUC of ACT 333679 by 27%.

Metabolism: Selexipag has five types of biotransformation reactions with the main pathway being the hydrolysis of the sulfonamide by carboxylesterases to form ACT 333679. It is important to also note that in the human, selexipag is metabolized to ACT 333679 from hepatic microsomes but carboxylases present in in plasma do not metabolize selexipag to ACT 333679.

Toxicology, Safety and Side Effects: Possible induction of gastrointestinal disturbances denoting intestinal intussusception (manifested as ileus or obstruction) induced by selexipag has been included as an important potential risk in the Risk Management Plan, and special caution is needed in the treatment of children, because of their higher susceptibility (EMA/272184/2016, page 22). Also ophthalmological side effects associated with the retinal vascular system have been included as an important risk in the Risk Management Plan. Other potential toxicities mentioned in the Uptravi Assessment Report include potential induction of neurogenic pain, headache, anthralgia, abdominal pain and pain in the jaw and extremities as well as induction of specific enzymes in the intestines. Side effects sorted by difference in incidence between selexipag and placebo and being higher than 10% include in order of higher incidence, headache, diarrhea, pain in the jaw, nausea and myalgia (Pulmonary Circulation Volume 7 No 3, p. 600). In general the important adverse events included various gastrointestinal symptoms, pain in various locations and flushing (vasodilation).

Transdermal Delivery: Transdermal delivery pertains to delivery of drugs through human skin and it thus encompasses both topical delivery in the form of gels, creams, and the like, applied directly to the skin as well as transdermal delivery in the form of patches. The patch systems can be classified in many ways, but they are mostly those that are called matrix patches where the active drug is incorporated in a polymer layer and the reservoir patches where the drug is a solution, a gel or a cream, enclosed between two or more polymer layers. In topical formulations as well as in reservoir type systems the gels or creams are formed by dissolving in the solutions small amounts of hydrophilic polymers, such as hydroxypropyl cellulose (e.g., KLUCEL™). Both patch systems are well accepted with transdermal products of both types available in the market place. Transdermal delivery has been used with different drugs and it is well understood, (Transdermal Delivery of Drugs, Volumes I, II and III, CRC Press, 1987) although drug permeation through skin and the increase of skin permeation by the use of chemical enhancers is still more of an art than science with many chemical enhancers used with hundreds of drugs, but with partial success (Drug Permeation Enhancers, Marcel Dekker, 1994). Chemical enhancers can be used with both topical formulations (U.S. Pat. No. 9,186,352) as well as with patch formulations (U.S. Pat. No. 9,198,919). Common chemical enhancers include DMSO, ethyl alcohol, lauryl lactate, ethyl lactate, capric acid, oleic acid, oleyl alcohol, glycerol monooleate, levulinic acid and dipropylene glycol among others. The book Percutaneous Penetration Enhancers, CRC Press, 1995, describes dozens of chemical families that can be used as enhancers and over 100 individual chemicals. The main component of the active portion of the patch is the pressure sensitive adhesive (PSA) into which the drug is dissolved or dispersed. Commercially used PSA adhesives include acrylic polymers and copolymers, silicones and polyisobutylenes and they represent anywhere from 50 to 95% of the drug active matrix. Acrylate PSA have great flexibility because of their ease in forming copolymers and allowing larger amounts of drugs to be incorporate within (U.S. Pat. No. 9,539,201). Because they are more hydrophilic than other PSA they are not commonly used for adhesion to skin for more than three and one half days. Polyisobutylene adhesives are the most hydrophobic and they are often used for the development of patches that adhere to the skin for seven days. These PSA are however not easy to modify, so in many cases the active portion of the patch, where the drug is dissolved, is made of acrylate adhesive and there is a peripheral adhesive attached to the back side of the patch and extending in all direction beyond the active patch to provide for long term adhesion of over seven days (U.S. Pat. No. 8,246,978). Humectants are also used in transdermal patches to absorb the transepidermal water loss and reduce irritation. Humectants are water soluble or swellable polymers and those more commonly used include polyvinyl pyrrolidone and polyvinyl pyrrolidone/vinyl acetate copolymers (U.S. Pat. Nos. 9,050,348; 9,539,201). Antioxidants are also used in the active portion of the patch if the drug is susceptible to oxidation. Oxidation can take place from oxygen permeating through the packaging film or from the inactive ingredients in the patch. For example acrylate pressure sensitive adhesives as well as polyvinyl pyrrolidone are manufactured by free radical polymerization processes. Therefore free radicals remaining within these polymers will degrade a susceptible drug when incorporated into these polymers (U.S. Pat. No. 9,364,487). Useful antioxidants include sodium bisulfite, sodium sulfite, isopropyl gallate, Vitamin C, Vitamin E, butylated hydroxyanisole (BHA), butylated hydroxytoluene (BHT), pentaerythritol tetrakis (3-(3,5-di-tert-butyl-4-hydroxyphenyl)propionate), or tris(2,4-di-tert-butylphenyl) phosphite.

### SUMMARY OF THE INVENTION

One aspect of the invention features a transdermal patch comprising ACT 333679 in a sufficient amount to deliver ACT 333679 for an extended period of time for the treatment of pulmonary arterial hypertension and/or other diseases of the vascular system. In one embodiment, the sufficient amount of time is one day or three and one half days or seven days.

The transdermal patch can be a matrix type patch or a reservoir type patch. In certain embodiments, the patch is covered by a peripheral pressure sensitive adhesive that extends beyond the patch in all directions. The peripheral adhesive can be a polyisobutylene pressure sensitive adhesive.

In certain embodiments, the patch comprises one or more other components selected from chemical enhancers, humectants, pressure sensitive adhesives, antioxidants, solubilizers, thickening agents, plasticizers, and any combinations thereof. The chemical enhancers can include one or more of DMSO, ethyl alcohol, oleic acid, oleyl alcohol, glycerol monooleate, levulinic acid, dipropylene glycol, diethylene glycol monoethyl ether, lauric lactate, ethyl lactate, and any combinations thereof. The humectant can be polyvinyl pyrrolidone or polyvinyl pyrrolidone/vinyl acetate copolymer. The pressure sensitive adhesive can be selected from a polyvinyl acetate polymer or copolymer pressure sensitive adhesive, or a silicone pressure sensitive adhesive or a polyisobutylene pressure sensitive adhesive.

Another aspect of the invention features other topical dosage forms comprising ACT 333679 for the delivery of therapeutic levels of ACT 333679 for an extended period of time for the treatment of pulmonary arterial hypertension and/or other diseases of the vascular system. These compositions for topical application include but are not limited to gel, ointment, emulsion, microemulsion, aqueous gel, foam, spray, lotion, or cream compositions. In certain embodiments, the extended period of time is one application every 4 hours or every 6 hours or every 8 hour or every 12 hours or every 24 hours.

The topical composition can comprise one or more other components selected from chemical enhancers, humectants, antioxidants, thickening agents, solubilizers, plasticizers, and any combinations thereof. The chemical enhancers can include one or more of DMSO, ethyl alcohol, oleic acid, oleyl alcohol, glycerol monooleate, levulinic acid, dipropylene glycol, diethylene glycol monoethyl ether, lauric lactate, and any combinations thereof. The thickening agent can be hydroxypropyl cellulose.

Another aspect of the invention features a method of administering ACT 333679 to an individual for the treatment of pulmonary arterial hypertension and/or another disease of the vascular system. The method comprises: (a) identifying an individual for whom administration of the ACT 33679 is indicated; and (b) topically applying to the individual one or more of (i) a transdermal patch comprising ACT 333679 in a sufficient amount to deliver ACT 333679 for an extended period; and/or (ii) a composition for topical application comprising ACT 333679 in a sufficient amount to topically deliver the ACT 333679 for an extended period.

In certain embodiments of the method, the individual is identified as one for whom administration of the ACT 33679 is indicated by exhibiting pulmonary hypertension and/or other indicia of disease of the vascular system.

In certain embodiments, the method includes testing the individual for reduction of the pulmonary hypertension and/or other indicia of remediation of disease of the vascular system before, after, and/or during the course of the administration period.

In one embodiment, the transdermal patch is applied to the individual and the sufficient amount of time is one day or three and one half days or seven days. In another embodiment, the composition for topical application is applied to the individual and the extended period is one application every 4 hours or every 6 hours or every 8 hour or every 12 hours or every 24 hours. These compositions for topical application include but are not limited to gel, ointment, emulsion, microemulsion, aqueous gel, foam, spray, lotion, or cream compositions.

Other features and advantages of the invention will be evident from the drawings, detailed description and examples that follow.

### BRIEF DESCRIPTION OF THE DRAWING

FIG. 1: Two graphs showing the in vitro permeation of selexipag (top panel) and ACT 333679 (bottom panel).

### DETAILED DESCRIPTION OF THE INVENTION

### Definitions

All percentages expressed herein are by weight of the total weight of the composition unless expressed otherwise. All ratios expressed herein are on a weight (w/w) basis unless expressed otherwise.

Ranges may be used herein in shorthand, to avoid having to list and describe each value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a method” or “a container” includes a plurality of such “methods”, or “containers.” Likewise the terms “include”, “including”, and “or” should all be construed to be inclusive, unless such a construction is clearly prohibited from the context. Similarly, the term “examples,” particularly when followed by a listing of terms, is merely exemplary and illustrative and should not be deemed exclusive or comprehensive.

The term “comprising” is intended to include embodiments encompassed by the terms “consisting essentially of” and “consisting of”. Similarly, the term “consisting essentially of” is intended to include embodiments encompassed by the term “consisting of.”

The methods and compositions and other advances disclosed herein are not limited to particular equipment or processes described herein because such equipment or processes may vary. Further, the terminology used herein is for describing particular embodiments only and is not intended to limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

The term “about” refers to the variation in the numerical value of a measurement, e.g., temperature, length, width, height, weight percentage, etc., due to typical error rates of the device used to obtain that measure. In one embodiment, the term “about” means within 5% of the reported numerical value.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### DESCRIPTION

Reviewing the selexipag literature, it became clear to me that there were a lot of serious issues with this drug such as, a) taking a drug orally that has major gastrointestinal side effects such as diarrhea, nausea, vomiting and intestinal intussusception, b) having important ophthalmological side effects such as retinal vascular system risk c) having a very short half-life which would necessitate the use of larger amounts of drug so as to prevent administering the drug every 4 or 6 hours, d) having rapid but low absorption in the area of 60% thus having a large portion of the drug excreted without performing its intended use, e) not being a very strong vasorelaxant and depending on its metabolite which is at least 16 times and up to 37 times more effective than selexipag itself and f) having patient to patient variability because of the enzymatic liver differences of different subjects which will affect the ratio of selexipag to metabolite in the plasma and thus the relative effectiveness.

Surprisingly it appeared that if the metabolite ACT 333679 could be delivered by transdermal administration it would eliminate or minimize most if not all of the selexipag issues. For example the issues associated with the short half-life will be eliminated because in transdermal delivery the drug is metered slowly and continuously so the half-life is almost of no consequence. The absorption of the drug administered transdermally will be 100% VS 60% orally. The metabolite will provide 16 to 37 percent higher effectiveness of the portion that in oral administration does not metabolize from selexipag to ACT 333679 (about 25 to 33%). Taking into account the advantages mentioned above it is the expectation that the relative amount of ACT 333679 that will have to be delivered transdermally to provide the same plasma concentration as selexipag will be 50% or less of the selexipag dosage administered orally. By using 50% less drug than used in oral delivery, it is expected that the gastrointestinal and ophthalmological side effects will be reduced, as well as some of the other side effects such as reduction in headaches, flushing, neurologic pain and pain of the abdomen, jaw and extremities.

Permeation of drugs through the skin is the most important factor when one considers transdermal delivery. Several articles have developed scientific rational and mathematical models to estimate the flux of a drug permeating through human skin (Models of Skin Permeability in Transdermal Delivery of Drugs, Vol III, p 41, CRC Press 1987; Effect of Lipid Solubility and Molecular Size on Percutaneous Absorption, in Skin Pharmacokinetics, S. Karger 1987) using basic physicochemical properties of the drug, such as molecular weight, melting point, partition coefficient and water solubility.

Using these basic properties for selexipag and ACT 333679 (Bioorganic and Medicinal Chemistry, 15 (2007) 6692) the estimated unenhanced permeation through skin was found to be for selexipag 25 micrograms per 10 cm² patch per day which is very small when compared to the effective dosage requirement mentioned above. Good enhancing systems could increase the permeation of selexipag, but still the flux values will be substantially lower than that of ACT 333679. In addition selexipag will need to be metabolized to ACT 333679 to be really effective and it is not guaranteed that the enzymes in the skin will be as effective as the enzymes in the liver in performing this hydrolytic biotransformation. In contrast the estimated skin permeation of ACT 333679 was 750 micrograms per 10 cm² patch per day, which meets and could easily surpass the dosage requirements mentioned above. In the examples below, the enhanced flux for ACT 333679 was shown to be 1600 and 3500 micrograms per 10 cm² per day. It is therefore an object of my invention to provide a transdermal patch comprising ACT 333679 without the need to use chemical enhancers. To decrease the size of the patch, chemical enhancers can be used, as has been mentioned above and shown in the examples below. Permeation enhancers are known to increase skin permeation by two to four-fold without affecting the skin such as causing itching or skin irritation. There is therefore another object of my invention to use chemical enhancers with patches, gels, ointments, emulsions, microemulsions, aqueous gels, foams, sprays, lotions or creams comprising ACT 333679 as to prepare patches and other topical dosage forms which have permeability through skin with better short term as well as long term adhesion and which are more cosmetically elegant.

### EXAMPLES

### Example 1. In Vitro Enhanced Skin Permeation of ACT 333679

One skin donor and three diffusion cells per formulation were used in these in vitro skin permeation experiments. Split thickness dermatomed (approximately at 375 μm) human cadaver skin was used to determine the permeation rate of the selexipag metabolite ACT 333679 in vitro. All in vitro skin permeation studies were conducted using the PermeGear Membrane Transport System (PermeGear, Inc., Hellertown, Pa.). Each Membrane Transport System consists of vertical, jacketed (37° C.±0.5° C.) Franz diffusion cells with magnetic stirrer and 1.7 cm² diffusion area.

Skin flux studies were run for a period of 48 hours. At predetermined intervals (3, 24 and 48 hours) after starting the experiment, the entire contents of the receiver compartment were collected for determination of the ACT 333679 concentration by HPLC. The receiver compartment was refilled with fresh receiver medium. The receiver medium was pH 7.4 water with 0.44 mg/ml of OLETH 20 (polyethylene glycol ether of oleyl alcohol) with the saturation concentration of the drug in the receptor medium being 0.23 mg/ml or 2 mg total in the receptor phase. This solubility of the drug in the receiver medium was sufficient to ensure sink conditions throughout each collection interval. The donor phase was composed of a saturated solution of ACT 333679 in a DMSO/ethanol/water solution and enhancers. Two formulations were prepared with different enhancer systems. Formulation A contained 8.3% each of the enhancers, oleic acid and levulinic acid and Formulation B contained 8.3% each of the enhancers, oleic acid and dipropylene glycol. The donor phase contained 0.2% ACT 333679.

The cumulative average permeation values in micrograms per square centimeter calculated for the two formulations were:

Formulation A, 3 hours-25; 24 hours-75; 48 hours-110.

Formulation B, 3 hours-130; 24 hour-350; 48 hours-400.

### Example 2. Selexipag Versus ACT 333679 In Vitro Enhanced Skin Permeation

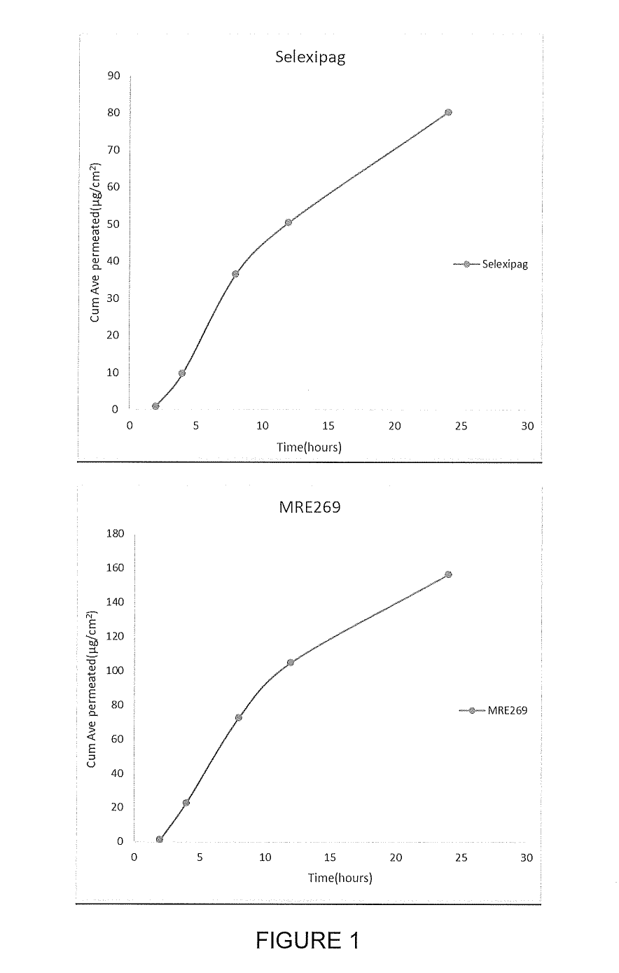

One skin donor and three diffusion cells per formulation were used in these in vitro skin permeation experiments. The experiment was performed using the same instruments and methodologies as described in Example 1. Two formulations were prepared containing selexipag and ACT 333679 respectively. Formulation B shown in example 1 was used with this experiment because the enhancing system appeared to be more effective. Samples in the receptor phase were obtained at the 2, 4, 8, 12 and 24 hour intervals and the amount of selexipag or ACT333679 was determined by HPLC. The permeation values are shown in FIG. 1. At all the time points the permeation through skin of the ACT 333679 was higher than that of selexipag.

### Example 3. In Vitro Unenhanced Skin Flux

One skin donor is used in these in vitro skin permeation experiments.

Split thickness dermatomed (approximately at 375 μm) human cadaver skin is used to determine the permeation rate of selexipag and metabolite ACT 333679 in vitro. All in vitro skin permeation studies are conducted using the PermeGear Membrane Transport System. Each Membrane Transport System consists of vertical, jacketed (37° C.±0.5° C.) Franz diffusion cells with magnetic stirrer.

Skin flux studies are run for a period of 168 hours. At predetermined intervals (24, 48, 72, 96, 120, 144, and 168 hours) after starting the experiment, the entire contents of the receiver compartment are collected for determination of the selexipag or ACT 333679 concentration by HPLC. The receiver compartment is refilled with fresh receiver medium. The solubility of the drugs in the receiver medium is sufficient to ensure sink conditions throughout each collection interval. The donor phase is composed of a saturated solution of each drug in an 80/20% ethanol/water solution.

The average flux values calculated for the 168 hour period for selexipag and ACT 333679 are respectively 1.2 and 2.9 micrograms per square centimeter per hour.

The present invention is not limited to the embodiments described and exemplified herein, but is capable of variation and modification within the scope of the appended claims.

What I claim is:

1. A transdermal patch comprising ACT 333679 in a sufficient amount to deliver ACT 333679 for an extended period of time for the treatment of pulmonary arterial hypertension and/or other diseases of the vascular system.

2. The transdermal patch of claim 1, wherein the sufficient amount of time is one day or three and one half days or seven days.

3. The transdermal patch of claim 1, comprising one or more other components selected from chemical enhancers, humectants, pressure sensitive adhesives, antioxidants, solubilizers, thickening agents, plasticizers, and any combinations thereof.

4. The transdermal patch of claim 1, wherein the patch is covered by a peripheral pressure sensitive adhesive that extends beyond the patch in all directions.

5. The transdermal patch of claim 4, wherein the peripheral adhesive is a polyisobutylene pressure sensitive adhesive.

6. The transdermal patch of claim 1, wherein the patch is a matrix patch.

7. The transdermal patch of claim 1, wherein the patch is a reservoir patch.

8. The transdermal patch of claim 3, wherein the chemical enhancers include one or more of DMSO, ethyl alcohol, oleic acid, oleyl alcohol, glycerol monooleate, levulinic acid, dipropylene glycol, diethylene glycol monoethyl ether, lauric lactate, ethyl lactate, and any combinations thereof.

9. The transdermal patch of claim 3, wherein the humectant is polyvinyl pyrrolidone or polyvinyl pyrrolidone/vinyl acetate copolymer.

10. The transdermal patch of claim 3, wherein the pressure sensitive adhesive is a polyvinyl acetate polymer or copolymer pressure sensitive adhesive, or a silicone pressure sensitive adhesive or a polyisobutylene pressure sensitive adhesive.

11. A composition for topical application comprising ACT 333679 for the delivery of therapeutic levels of ACT 333679 for an extended period of time for the treatment of pulmonary arterial hypertension and/or other diseases of the vascular system.

12. The composition of claim 11, formulated as one or more of a gel, ointment, emulsion, microemulsion, aqueous gel, foam, spray, lotion or cream.

13. The composition of claim 11, comprising one or more other components selected from chemical enhancers, humectants, antioxidants, thickening agents, solubilizers, plasticizers, and any combinations thereof.

14. The composition of claim 13, wherein the chemical enhancers include one or more of DMSO, ethyl alcohol, oleic acid, oleyl alcohol, glycerol monooleate, levulinic acid, dipropylene glycol, diethylene glycol monoethyl ether, lauric lactate, and any combinations thereof.

15. The composition of claim 13, wherein the thickening agent is hydroxypropyl cellulose.

16. The composition of claim 11, wherein the extended period of time is one application every 4 hours or every 6 hours or every 8 hour or every 12 hours or every 24 hours.

17. A method of administering ACT 333679 to an individual for the treatment of pulmonary arterial hypertension and/or another disease of the vascular system, comprising:
a. Identifying an individual for whom administration of the ACT 33679 is indicated; and
b. topically applying to the individual one or more of
i. a transdermal patch comprising ACT 333679 in a sufficient amount to deliver ACT 333679 for an extended period; and
ii. A composition for topical application comprising ACT 333679 in a sufficient amount to topically deliver the ACT 333679 for an extended period.

18. The method of claim 17, wherein the individual is identified as one for whom administration of the ACT 33679 is indicated by exhibiting pulmonary hypertension and/or other indicia of disease of the vascular system.

19. The method of claim 17, comprising testing the individual for reduction of the pulmonary hypertension and/or other indicia of remediation of disease of the vascular system before, after, and/or during the course of the administration period.

20. The method of claim 17, wherein:
a. the transdermal patch is applied to the individual and the sufficient amount of time is one day or three and one half days or seven days; or
b. the composition for topical application is applied to the individual and the extended period is one application every 4 hours or every 6 hours or every 8 hour or every 12 hours or every 24 hours.
